SEQUENCE LISTING (36 sequences)

SEQ ID NO: 1 - DNA, 31 bp, Artificial Sequence
  Description of Artificial Sequence gene specific oligonucleotide for PCR
gtccacggtctcccatgcagaatttcgagat

SEQ ID NO: 2 - DNA, 26 bp, Artificial Sequence
  Description of Artificial Sequence gene specific oligonucleotide for PCR
acatcccaagcttcacgttcgaggtc

SEQ ID NO: 3 - DNA, 27 bp, Artificial Sequence
  Description of Artificial Sequence gene specific oligonucleotide for PCR
cgctcggtcatgacgtacgtttccctg

SEQ ID NO: 4 - DNA, 28 bp, Artificial Sequence
  Description of Artificial Sequence gene specific oligonucleotide for PCR
acgaagaagcttatcaaacaacccccag

SEQ ID NO: 5 - DNA, 26 bp, Artificial Sequence
  Description of Artificial Sequence gene specific oligonucleotide for PCR
atgccgcccagtgacatcgcgtacgc

SEQ ID NO: 6 - DNA, 33 bp, Artificial Sequence
  Description of Artificial Sequence gene specific oligonucleotide for PCR
ccctcggtaccaggtcatcggccggccacttcc

SEQ ID NO: 7 - DNA, 24 bp, Artificial Sequence
  Description of Artificial Sequence gene specific oligonucleotide for PCR
atgcgggagccgctcacgctcgac

SEQ ID NO: 8 - DNA, 36 bp, Artificial Sequence
  Description of Artificial Sequence gene specific oligonucleotide for PCR
ccaactggtacctggtcatccgtggctcctgtctcg

SEQ ID NO: 9 - DNA, 1140 bp, Nocardia uniformis
  CDS (52)..(1086): CDS for p-hydroxymandelate synthase from Nocardia uniformis subsp. tsuyamanensis ATCC 21806
aaaccgtctataaatgctgccggagggcgatcccacggctggaggttcgtgatggcggcacaggcaggcagcgtgttcgacggcatgacgctcgaccacaccgtgttctacgtcggcgacgcggaccgcgcggcgggcgagctcaccgacaagtacgggctggtggtgctcggcacgtccgagacctcgtcggtgcgctcggtcgcggtgggcggcggctccatccggctggtgttctcccaggccatcgccgacgacaccccggctgccgcgtacgtgaaggtgcacggcgacggcgtggccgacctcgcgctgggcgtcgccgacgcccgcgccgcgttcgccgaggcggtgcggcggggcgcgcggccggtggccgagcccaccgaggccgacggcgcggtgctcgccacgatcatgggcttcggcgacgtggtgcacaccttcgtccagcgccccggcggcgcgcccggcgaggacccggagagcgcgggcggcctgcgcgtgctggaccacttcgcggtctgcctcgaggcgggcgggctggagccgaccgtggcgttctaccaggaggtgctggacttccgggtggtcttcgaggagaagatcgtcgtcggggcgcaggcgatgaactccaaggtcgtgcagagcacgtccggcgcggtcacgctgaccctcatcgaaccggacacctcgcgcaagcccggtcagatcgacgacttcatcaagaaccacggtggcgcgggcgtgcagcacatcgccttcgccaccgacggcatcgtggacgcggtgcgccggttgcgcgagcggggcgtggagctgctcaccacgcccgccgcctactacgacctgctggcggaccgcctcggaccgacccggtactccacggccgagctggccgagctgaacctgctggtcgacgaggaccaggacggcaagctgtaccagatcttcgcccggtccacccaccccaggggcacgttcttcttcgagatcatcgagcgcgcgggcgcgcacaccttcggcagcggcaacatcaaggccctctacgaggccgtcgaggccgagcgccaccggaccgagcgctgagcgcggggcacggcgatggacccgctgcgcgccggtgacctgcacggctcg

SEQ ID NO: 10 - protein, 345 aa, Nocardia uniformis
MAAQAGSVFDGMTLDHTVFYVGDADRAAGELTDKYGLVVLGTSETSSVRSVAVGGGSIRLVFSQAIADDTPAAAYVKVHGDGVADLALGVADARAAFAEAVRRGARPVAEPTEADGAVLATIMGFGDVVHTFVQRPGGAPGEDPESAGGLRVLDHFAVCLEAGGLEPTVAFYQEVLDFRVVFEEKIVVGAQAMNSKVVQSTSGAVTLTLIEPDTSRKPGQIDDFIKNHGGAGVQHIAFATDGIVDAVRRLRERGVELLTTPAAYYDLLADRLGPTRYSTAELAELNLLVDEDQDGKLYQIFARSTHPRGTFFFEIIERAGAHTFGSGNIKALYEAVEAERHRTER

SEQ ID NO: 11 - DNA, 26 bp, Artificial Sequence
  Description of Artificial Sequence gene specific oligonucleotide for PCR
agaattcgcggcacaggcaggcagcg

SEQ ID NO: 12 - DNA, 30 bp, Artificial Sequence
  Description of Artificial Sequence gene specific oligonucleotide for PCR
ttataagctttcagcgctcggtccggtggc

SEQ ID NO: 13 - DNA, 24 bp, Artificial Sequence
  Description of Artificial Sequence gene specific oligonucleotide for PCR
tataccatggcggcacaggcaggc

SEQ ID NO: 14 - DNA, 27 bp, Artificial Sequence
  Description of Artificial Sequence gene specific oligonucleotide for PCR
ttataagcttgcgctcggtccggtggc

SEQ ID NO: 15 - DNA, 1227 bp, Nocardia uniformis
  CDS (50)..(1177): CDS for p-hydroxymandelate oxidase from Nocardia uniformis subsp. tsuyamanensis ATCC 21806
ccccgcgcgtggggaggcggcgtgcccgacggacaggaggcaacgagccatgggcgtccgcaactccgcagggggcggcgcggaagaccccgaggacctggctgaggtggaacgggccgccgccgcccggctgccgggggacgtgcgcgacttcatcgcgggcggcagcggcgacgaggtgacgctggccgccaaccgcgcggcgctcgacgacgtggccctgctccccagggtgctcgcgggtgtccaggcggccgacacgagcacctcgctcgtgggcacggcggccacgctgcccgtcgccgtggcgccgatgggctaccagtgcctcgtccaccccgacggcgaggtcgcggctgccgccgcggcgggcgccgccggcgtccccttcaccgtcggcacgctgagcagccggtcggtcgaggagatcgccgagaccggcgcgtcgctgtggttccagctctactggctgcgcgatcgcggcctggtcgccgaactcgtggcgcgggccgaggcggcgggctgccgggcgctggtgatcaccgtggacgtgccggtgatgggccgcaggctccgggacgtgcgcaacgggatcaccctgccccggaccgtccgggccgtccacctcgccgacggcccgtcatccgcgcacgagccgcgccaggtcggctccggcgtcgcccagcacacgagcgcggtcttcgaccccgcgttcgggtggcgcgacctggagtggctgcgggcgcgcaccaggctccccctggtggtcaagggcgtgctcgacccgcgcgacgccaccaggtgcgtcgagctgggcgcctcggcggtggtggtgtccaaccacggcgggcggcagctcgacggcgcggcgcccagcgcggtggccctgccgcgcgtcgtcgacgcggtggcgggcgcggccgaggtgctgttcgacagcggcgtccgcggcggcgtcgacgtgctgcgcgccctcgcgctcggcgcgaccggcgtgctgctcggccgcccaatcctgtgggggctcgcggtcggcggcgagcgcggcgcggcgcgggtgctggaactgctgcgcaccgagttcgcgcaggccctgctgctcgccgggtgcgccgacgtcgacgcggccaggggactcgcgaccgcgcaggccgcgccgacccggcgaggtgcgccctgaccgcctcggggaccgcgatcggggccggggcgcggggtcaggggaag

SEQ ID NO: 16 - protein, 376 aa, Nocardia uniformis
MGVRNSAGGGAEDPEDLAEVERAAAARLPGDVRDFIAGGSGDEVTLAANRAALDDVALLPRVLAGVQAADTSTSLVGTAATLPVAVAPMGYQCLVHPDGEVAAAAAAGAAGVPFTVGTLSSRSVEEIAETGASLWFQLYWLRDRGLVAELVARAEAAGCRALVITVDVPVMGRRLRDVRNGITLPRTVRAVHLADGPSSAHEPRQVGSGVAQHTSAVFDPAFGWRDLEWLRARTRLPLVVKGVLDPRDATRCVELGASAVVVSNHGGRQLDGAAPSAVALPRVVDAVAGAAEVLFDSGVRGGVDVLRALALGATGVLLGRPILWGLAVGGERGAARVLELLRTEFAQALLLAGCADVDAARGLATAQAAPTRRGAP

SEQ ID NO: 17 - DNA, 26 bp, Artificial Sequence
  Description of Artificial Sequence gene specific oligonucleotide for PCR
agaattcggcgtccgcaactccgcag

SEQ ID NO: 18 - DNA, 26 bp, Artificial Sequence
  Description of Artificial Sequence gene specific oligonucleotide for PCR
aataagctttcagggcgcacctcgcc

SEQ ID NO: 19 - DNA, 23 bp, Artificial Sequence
  Description of Artificial Sequence gene specific oligonucleotide for PCR
actcgccaagggctatggtgtcc

SEQ ID NO: 20 - DNA, 24 bp, Artificial Sequence
  Description of Artificial Sequence gene specific oligonucleotide for PCR
gccaacagttccaacagcggtgtg

SEQ ID NO: 21 - DNA, 1427 bp, Pseudomonas putida
  CDS (51)..(1376): CDS for D-p-hydroxyphenylglycine aminotransferase from Pseudomonas putida NCIMB 12565
tcagttaactgccatgtaatctcaaaaaccagtccaatagaggcatcagcatgtctatttatagcgattatgaacgtaaaacggtgggctccgcgcattgggcggatcgggcacgagctgtaatgccagatggcgtaacggctgatacacgtgtttttgatccgcatgggctgtacatctgtgacgcgcaaggtgttcacaagaccgatgttgatggcaacgtatacctggatttttttggtgggcatggcgctttagtgcttggtcatggacatccacgtattaacaaggcaatctctgcagcgctgacgcatggcgtccaatacggtgcaagccatccgcttgaagtgcgatgggccgagcgcttggtcaacgccttcccttcgatgcataaagtgcgctttgccggcagcggcactgaggcgactttattggcactgcgaatggcgcgggcgtttactggcaggtcgaaaatactacgtattgccacccactatcatggctggcatgacttctccgcctctggttacaacagtcacttcgacggccagccagcaccgggcgtccttgctgaaattgcacagagcacactactggttcgcccagatgacttcgaaggattacgcgcactattcgctcaatacggcggtgagattgcgaccattattgcggagccagtcggctctcatttcggtatcactccggtcagcgacgatttcttgctagaaggcgccgcgctggctcgaaagcacggtgcaatattcattcttgacgaagtgataaccggttttcgcgtgggtaaccacggtatgcaggggcttctcgacattgcaccggatcttacgtgcttggccaaggccagtgctggaggtcttcctggaggcgtagtcggaggaagggcggatgtcatggcggtactggatcgcggttcagaccgtaaggttctgcatcaaggcaccttcaccggaaacccaattacggcggcggcagcaatcgcggcgatcgataccattatcgaagatgacgtgtgcacacacatcaatgccctcggtcaatacgcccgggactcgatgaacgaactcttcgctcgtaagcatttggactggttggcttatggccgcttttccggcttccacctgatgccgggattatctccgctgaccactgacacgggtgtgatcactcgcggagaagttgcacggcccgcagtaaaaatgattgccgctatgcgcatggcattaatccttgagggcatcgatattggtggccgtggatctgtgtttctctctgcgcagcatgatcgcggacatgtcgaccaactggtgtcgacctttgattccattcttggccgcctggctgaagaaaacctcctgggctaagcccttacacatctgtctggaatatgaatcatgaaaactagcctgaaa

SEQ ID NO: 22 - protein, 442 aa, Pseudomonas putida
MSIYSDYERKTVGSAHWADRARAVMPDGVTADTRVFDPHGLYICDAQGVHKTDVDGNVYLDFFGGHGALVLGHGHPRINKAISAALTHGVQYGASHPLEVRWAERLVNAFPSMHKVRFAGSGTEATLLALRMARAFTGRSKILRIATHYHGWHDFSASGYNSHFDGQPAPGVLAEIAQSTLLVRPDDFEGLRALFAQYGGEIATIIAEPVGSHFGITPVSDDFLLEGAALARKHGAIFILDEVITGFRVGNHGMQGLLDIAPDLTCLAKASAGGLPGGVVGGRADVMAVLDRGSDRKVLHQGTFTGNPITAAAAIAAIDTIIEDDVCTHINALGQYARDSMNELFARKHLDWLAYGRFSGFHLMPGLSPLTTDTGVITRGEVARPAVKMIAAMRMALILEGIDIGGRGSVFLSAQHDRGHVDQLVSTFDSILGRLAEENLLG

SEQ ID NO: 23 - DNA, 46 bp, Artificial Sequence
  Description of Artificial Sequence gene specific oligonucleotide for PCR
gtgcacggtctcgcatgtctatttatagcgattatgaacgtaaaac

SEQ ID NO: 24 - DNA, 42 bp, Artificial Sequence
  Description of Artificial Sequence gene specific oligonucleotide for PCR
gtgcacggtctcctcgagttagcccaggaggttttcttcagc

SEQ ID NO: 25 - DNA, 37 bp, Artificial Sequence
  Description of Artificial Sequence gene specific oligonucleotide for PCR
gggaattcaggaggaattaaccatgccgccgagcgac

SEQ ID NO: 26 - DNA, 37 bp, Artificial Sequence
  Description of Artificial Sequence gene specific oligonucleotide for PCR
gaattcccatattctagaaggtcatcggccggccact

SEQ ID NO: 27 - DNA, 29 bp, Artificial Sequence
  Description of Artificial Sequence gene specific oligonucleotide for PCR
tgggaattcaggaggaattaaccatgcag

SEQ ID NO: 28 - DNA, 27 bp, Artificial Sequence
  Description of Artificial Sequence gene specific oligonucleotide for PCR
cggccaggtctagatacgtcatcgccg

SEQ ID NO: 29 - DNA, 35 bp, Artificial Sequence
  Description of Artificial Sequence gene specific oligonucleotide for PCR
tgggtctagaggaggaattaaccatgcgcgagccg

SEQ ID NO: 30 - DNA, 35 bp, Artificial Sequence
  Description of Artificial Sequence gene specific oligonucleotide for PCR
gaattcccatagcatgcctggtcatccgtggctcc

SEQ ID NO: 31 - DNA, 31 bp, Artificial Sequence
  Description of Artificial Sequence gene specific oligonucleotide for PCR
tttcccaagcttacaggaggaattaaccatg

SEQ ID NO: 32 - DNA, 30 bp, Artificial Sequence
  Description of Artificial Sequence gene specific oligonucleotide for PCR
gtaccagctgcaaagcttgagttagcccag

SEQ ID NO: 33 - DNA, 40 bp, Artificial Sequence
  Description of Artificial Sequence gene specific oligonucleotide for PCR
gggtctagaggaggaattaaccatgagccagaatctcttt

SEQ ID NO: 34 - DNA, 39 bp, Artificial Sequence
  Description of Artificial Sequence gene specific oligonucleotide for PCR
ctgcagaaccagcatggtggtcagtacttcactcatgcg

SEQ ID NO: 35 - DNA, 36 bp, Artificial Sequence
  Description of Artificial Sequence gene specific oligonucleotide for PCR
gcgtggaagcttaagaggtttattatggttgctgaa

SEQ ID NO: 36 - DNA, 40 bp, Artificial Sequence
  Description of Artificial Sequence gene specific oligonucleotide for PCR
gtgcacggtctcgagctgaattcttactggcgattgtcat

(19) United States
(12) Patent Application Publication
(10) Pub. No.: US 2005/0089974 A1
(43) Pub. Date: Apr. 28, 2005

(54) Fermentative production of d-hydroxyphenylglycine and d-phenylglycine

(76) Inventors: Craig A. Townsend, Baltimore, MD (US); Michele Gunsior, Owings Mills, MD (US); Ulrike Muller, Linnich-Rurdorf (DE); Friso B J Assema Van, Geleen (NL); Theodorus Sonke, Guttecoven (NL)

Correspondence Address: PILLSBURY WINTHROP, LLP, P.O. BOX 10500, MCLEAN, VA, 22102, US

(21) Appl. No.: 10/399,693

(22) PCT Filed: Oct. 22, 2001
(86) PCT No.: PCT/NL01/00772

(30) Foreign Application Priority Data
Oct. 27, 2000 (US) 09697769

(51) Int. Cl. C12P 13/22

(52) U.S. Cl. 435/108

(57) ABSTRACT

A new fermentative process for the preparation of D-p-hydroxyphenylglycine (D-HPG) or D-henylglycine (D-pG) in enantiomerically pure form is disclosed. Precursors for the formation of D-HPG and D-pG are withdrawn form the common aromatic amino acid pathway, converted to p-hydroxyphenylglyoxylate or phenylglyoxylate, and are finally converted to D-HPG or D-pG by the action of a stero-inverting D-aminotransferase.

This invention was made with German Government support under Grant No. 0311644 awarded by the BioRegio program of the Bundesministerium für Bildung und Forschung (BMBF), and with U.S. Government support under Grant No. RO1 A114937 awarded by the National Institutes of Health. The U.S. Government has certain rights in this invention.

### FIELD OF THE INVENTION

The invention relates to a biochemical process for the preparation of D-p-hydroxyphenylglycine (D-HPG) and D-phenylglycine (D-pG) in enantiomerically pure form. The invention also relates to recombinant microorganisms for the production of D-HPG and D-pG. Hereinafter the abbreviation (H)PG refers to HPG and/or PG; where required the specific enantiomer of (H)PG is mentioned.

### BACKGROUND OF THE INVENTION

Except for glycine, each of the common, naturally occurring amino acids can exist as one of two possible enantiomers. The two enantiomeric forms of an amino acid can be referred to as the D- and the L-configured enantiomer. The enantiomerical purity of compounds which may exist in two enantiomeric forms is generally expressed in terms of its enantiomeric excess (often abbreviated as “e.e.”); e.e. can be defined as the difference in the amounts of the two enantiomers divided by the sum of those amounts, the quotient being multiplied by 100 to get a percentage value. In the context of this application enantiomerically pure will mean an e.e. of at least 90%, preferably more than 95%, or even more than 98%.

The distinction between the D- and the L-configured enantiomer is made upon whether the conformation of the α-carbon of the amino acid corresponds to the L- or D-form of glyceraldehyde, an arbitrary standard. Most enzymes which act upon amino acids have L-specific binding domains, thus, most naturally occurring proteins comprise only L-amino acids.

There are, however, a few exceptions in which D-amino acids are used by and built into microbial cells. Bacterial cells, for example, produce D-glutamate and D-alanine as a precursor material for murein, a typical cell wall component. D-amino acids are typically not produced directly, but rather by conversion of the L-amino acid to the corresponding D-amino acid by the use of an amino acid specific racemase. Racemases catalyze the conversion of L-amino acids to D-amino acids and vice versa. In equilibrium, one finds a racemic (50%/50%) mixture of L- and D-enantiomers, hence, racemases cannot be used as a final reaction step for the production of D-amino acids in enantiomerically pure form.

An increasing demand for antibacterial drugs has led to a mass production of semisynthetic antibiotics, many of which have incorporated optically active D-amino acids as building blocks. D-HPG and D-pG, e.g., are incorporated in some β-lactam antibiotics, such as amoxicillin and cephalexin.

For cost effective production of these antibiotics, it is required to have available a commercially attractive process for the preparation of D-(H)PG in enantiomerically pure form. However, due to their complex structure and due to the occurrence of a chiral center within the molecules, the production of D-(H)PG with conventional chemical methods generally involves multiple steps for the production of a racemic mixture of D- and L-configured (H)PG and then requires additional optical resolution steps to obtain the products in enantiomerically pure form. The additional process steps add to the process costs and render the overall process commercially less attractive.

Fermentative processes for the production of fine chemicals are generally known as being commercially attractive for their ability to convert relatively cheap starting material to the product. The reactions involved in fermentative processes are usually highly regio- and stereoselective. They therefore allow the production of complex and enantiomerically pure products in relatively simple processes. For this reason, numerous attempts have been made in the prior art to produce various fine chemicals by fermentation.

Methods for the production of D-phenylalanine in a fermentative process using an E. coli strain containing a Bacillus sphaericus stereo-conserving D-aminotransferase, have been described (EP 0736604A2). “Stereo-conserving D-aminotransferases” shall be understood as D-aminotransferases that exclusively produce D-configured amino acids from D-configured amino donors. “Stereo-inverting D-aminotransferases”, on the other hand, shall be defined as aminotransferases which use L-configured amino donors and exclusively produce D-configured amino acids. For instance, L-Glu or L-Asp can be used as amino donor in the production of D-pG or D-HPG.

In order to provide the D-configured amino donor for the stereo-conserving D-specific aminotransferase reaction in vivo, genes coding for racemases which catalyze the conversion of L-amino acid to D-amino acid and vice versa, must be present in the cell. Hence, the D-configured amino donor is produced intracellularly from the L-configured enantiomer. The intracellular concentration of the D-configured amino donor will be half or less than half of the total intracellular concentration of said molecule (D- plus L-form). Because decreasing concentrations of the D-configured amino donor (an educt) lead to a shift of the equilibrium of the transamination reaction towards the educt side, a racemic mixture of the amino donor (i.e. the use of racemases in combination with stereo-conserving aminotransferases) is unfavorable for the efficient production of the desired D-amino acid.

Additionally, cloning a racemase into the cell is costly in labor and it increases the “metabolic burden” of the cell in that the cell has to synthesize an additional enzyme. The precursor metabolites and the energy consumed for the biosynthesis of the racemase are no longer available for cell growth, product formation, etc.

The aim of the current invention is to make available an improved fermentative process for the production of D-HPG and D-pG, which overcomes the above mentioned shortcomings.

This aim, surprisingly, is achieved in that in the fermentative process phenylpyruvate (PP) or p-hydroxyphenylpyruvate (HPP) are withdrawn from the aromatic amino acid pathway and converted to mandelic acid (MA) or p-hydroxymandelic acid (HMA), respectively, MA or HMA being converted to phenylglyoxylate (PG L) or p-hydroxyphenylglyoxylate (HPGL), respectively, and finally converted to D-pG or D-HPG, respectively, by the action of a stereo-inverting aminotransferase.

The application of a stereo-inverting aminotransferase in a fermentative process provides the clear advantages that no racemase activity is required and that the stereo-inverting aminotransferase uses naturally occurring L-configured amino donors (which are present intracellularly in at least twice the concentration of the respective D-enantiomer, when said D-enantiomer was produced from the L-enantiomer by a racemase). The application of a fermentative process allows the utilization of cheap starting materials such as glucose and other sugars for the production of D-(H)PG.

Fermentation (and a fermentative process), within the meaning of the invention, shall be understood as the process of cultivating a microorganism in a suitable medium, converting components of a suitable medium to the product by the catalytic activity of cellular components or by spontaneous chemical reactions, and obtaining the product from the cultivation broth and/or from the biomass itself.

A suitable medium, within the meaning of the invention, shall be understood as a mixture of substances on which the microorganism can grow, proliferate, and/or which can be converted to the desired product.

In a preferred embodiment of the invention, the microorganism applied in the fermentation is selected from a group of microorganisms which replenish HPP and/or PP (in cases where HPP and/or PP are withdrawn from the respective metabolite pool) more efficiently than typically observed in wild type strains. A microorganism selected from said group of microorganisms shall be referred to as having “improved production capabilities” for HPP and/or PP, and to “provide HPP and/or PP at increased availability”.

The application of a microorganism that provides HPP and/or PP at increased availability is important for the efficient production of D-HPG or D-pG because in a sequence of reactions not only the slowest reaction of the sequence but every enzymatic reaction controls the production rate to a certain extent. Hence, also the conversion from glucose to HPP and/or PP has an influence on the total production of the final product D-HPG or D-pG.

Suitable measures to increase the availability of HPP and PP in said microorganisms comprise the selection of microorganisms showing a beneficial spontaneous mutation, the selection of microorganisms that evolve from classical strain improvement programs involving random mutagenesis, and the selection of microorganisms with improved production capabilities that evolved from application of recombinant DNA technology. Suitable measures to increase the availability of HPP and PP, e.g., can be the introduction of feedback resistant mutants of key enzymes or the overexpression of one or several pathway enzymes as reviewed by Berry (1996, TIBTECH, 14: 250-256). Application of recombinant DNA technology can also be directed towards the introduction of phosphoenolpyruvate-independent sugar uptake systems (WO 98/18936), towards the deletion of the phosphotransferase system (PTS) (Berry, 1996, TIBTECH, 14: 250-256) and/or towards an increased availability of phosphoenolpyruvate by modifications of reactions acting on the intracellular phosphoenolpyruvate concentration (Berry, 1996, TIBTECH, 14: 250-256). Application of recombinant DNA technology can also be directed towards introducing increased intracellular transketolase and/or transaldolase activity (WO 98/18936).

Another suitable measure to provide HPP and PP at increased availability is to supply L-tyrosine, respectively L-phenylalanine, in the fermentation medium. L-Tyrosine is readily converted to HPP, whereas L-phenylalanine is readily converted to PP, by the microorganism in a single aminotransferase reaction step.

In a preferred embodiment, microorganisms according to the invention, convert PP to MA and/or convert HPP to HMA in a single-step enzymatic conversion catalyzed by phydroxymandelate synthase (p-HmaS). The conversion of HPP to HMA by an enzyme named p-HmaS was described by Choroba et al. (2000, J. Am. Chem. Soc., 122 (22): 5389-90).

The advantage of using a single enzyme for the conversion of HPP or PP to HMA or MA is that it is usually easier to clone and establish the in vivo activity of a single enzyme as compared to cloning and establishing the activity of multiple enzymes performing the same overall conversion.

Alternatively, a microorganism according to the invention converts PP to MA and/or HPP to HMA in a sequence of reactions comprising the reactions from PP or HPP for instance to phenylacetaldehyde or p-hydroxyphenylacetaldehyde to phenylacetate or p-hydroxyphenylacetate to MA or HMA, respectively. This sequence of reactions is deemed a possible metabolic route as it was observed in the degradation of phenylalanine in a basidiomycete by Krings et al. (1996, Journal of Biotechnology, 51: 123-129)).

Microorganisms according to the invention, irrespective of the way in which they produce HMA or MA, convert MA to PGL and/or HMA to HPGL by the enzymatic activity of a mandelate dehydrogenase and/or a phydroxymandelate dehydrogenase, respectively. Alternatively, said microorganisms convert MA to PGL and/or HMA to HPGL by the enzymatic activity of an oxygen dependent mandelate oxidase and/or an oxygen dependent phydroxymandelate oxidase, respectively. Microorganisms according to the invention are furthermore capable of converting HPGL, respectively PGL, to D-HPG, respectively to D-pG, by the action of a stereo-inverting aminotransferase.

The present invention also relates to the production of D-(H)PG by culturing said recombinant cells in a suitable medium and obtaining the product from the culture broth or from the microorganism itself.

The present invention also relates to a recombinant microorganism (i.e. to a recombinant cell) which is capable of secreting detectible amounts of D-HPG and/or D-pG and contains genes, coding for enzymes which catalyze in a single step, and/or by combined and subsequent action, the conversion of HPP to HMA and/or of PP to MA, the conversion of HMA to HPGL and/or of MA to PGL, and the conversion of HPGL to D-HPG and/or of PGL to D-pG, the final step being catalyzed by the action of a stereo-inverting D-aminotransferase.

It is to be noticed that the production of D-pG and D-HPG using a stereo-inverting aminotransferase is described by Wiyakrutta and Meevootisom (1997, Journal of Biotechnology, 55: 193-203). However, a whole cell fermentative approach is not considered. Instead, the authors mention reactions with purified enzyme. Furthermore, Taylor et al. (1998, TIBTECH, 16: 412-418) mention that the educts, PGL and HPGL, are very expensive, as compared to the value of the final products, D-pG and D-HPG. Thus, these references do not suggest any feasible application of a stereo-inverting aminotransferase for the production of D-HPG or D-pG.

Moreover, combination of a fermentative pathway to HPGL or PGL with the enzymatic activity of a stereo-inverting D-aminotransferase in vivo would have been expected to have limited chances of success because of incompatible pH optima of the respective enzymes. The intracellular pH of E. coli cells is reported to be in the range of pH 7.4 to pH 7.8 (Neidhardt et al., 1996, Escherichia coli and Salmonella typhimurium. Cellular and molecular biology., volume 1, chapter 96, page 1539, ASM Press, Washington, D.C.). The stereo-inverting aminotransferases, however, as reported by Wiyakrutta and Meevootisom (see above) have pH optima at pH 9 to pH 10 and very little activity at neutral pH. Thus, one would expect that a stereo-inverting aminotransferase does not have sufficient in vivo activity in E. coli cells to produce D-HPG or D-pG effectively.

Apart from an incompatibility of pH optima, several other problems can be expected, when enzymes from different organisms are combined to a new metabolic pathway in a host cell. The affinity of newly introduced enzymes might be very different from the intracellular metabolite concentrations. Furthermore, the stability of the heterologous enzyme might be low due to a high susceptibility of heterologous proteins towards intracellular proteases. The intermediates of the newly constructed pathway themselves might be unstable or metabolized by native enzymes of the host cell. Problems in the functional expression of the heterologous enzymes can also arise from inappropriate folding of correctly translated amino acid chains or from a difference in codon usage which might hinder the effective functional expression of the heterologous enzyme.

Despite the numerous problems that are expected in the construction of the new metabolic pathway, surprisingly, we found that D-HPG and D-pG were produced by recombinant microorganisms in a process according to the invention.

Finally, the invention also relates to recombinant plasmids containing at least one gene having a sequence corresponding with any of [SEQ ID: No.9] and/or [SEQ ID: No.15] and/or [SEQ ID: No.21], as disclosed hereinafter in this application, or homologues of any of these sequences having at least 80%, preferably at least 90%, most preferably at least 95% homology with any of such sequences.

Other features of the invention will become apparent in the course of the following descriptions of the examples.

General Procedures

Standard molecular cloning techniques such as plasmid DNA isolation, gel electrophoresis, enzymatic restriction modification of nucleic acids, E. coli transformation etc. were performed as described by Sambrook et al., 1989, “Molecular Cloning: a laboratory manual”, Cold spring Harbor Laboratories, Cold Spring Harbor, N.Y. Synthetic oligodeoxynucleotides were obtained from MWG-Biotech AG (Ebersberg, Germany), Sigma-Genosys (The Woodlands, Tex., USA), and Life Technologies (Paisley, Scotland, UK). DNA sequence analyses were performed by BaseClear (Leiden, The Netherlands), GATC Biotech AG (Konstanz, Germany), and Johns Hopkins University School of Medicine Biosynthesis and Sequencing Facility (Baltimore, Md., USA) using the chain termination method with dye-labeled dideoxy-terminators. Protein concentrations in crude extracts were determined with Bradford reagent at 595 nm (Roth, Karlsruhe, Germany) according to the instructions of the supplier.

### EXPERIMENTAL PART I (EXAMPLES 1-19)

### Cloning and Verification of Encoded Activity of Relevant Single Genes

### Example 1

### Construction of Plasmids pBAD-Ao-HmaS, pBAD-Ao-HmaO, pBAD-Sc-HmaS, pBAD-Sc-HmaO

Amycolatopsis orientalis NRRL 18098 (U.S. Pat. No. 5,843,437) was obtained from the ARS (Agricultural Research Service) Patent Culture Collection, Peoria, Ill., USA.

A. orientalis was cultivated in 1% glucose, 0.5% yeast extract (Difco, Detroit, Mich., USA), 2% starch, 0.1% casamino acids (Difco), pH 7.5 with NaOH at 28° C. Streptomyces coelicolor A3(2), kindly obtained from Professor M. J. Bibb of John Innes Institute, Norwich (UK), was cultivated in YE-ME medium containing 3 g/l yeast extract (Difco), 5 gA peptone (Difco), 3 g91 malt extract (Oxoid, Basingstoke, UK), 10 g9/glucose, 340 g/l sucrose at 28° C. 10 g/l glycine and 5 mM MgCl₂ were added after sterilisation.

The genomic DNA from A. orientalis and S. coelicolor was isolated by a salting out procedure (Pospiech and Neumann, 1995. Trends Genet. 11: 217-218).

The HmaS and HmaO genes of S. coelicolor and A. orientalis were cloned in expression vector pBAD/Myc-HisC (Invitrogen, Groningen, The Netherlands). Genes were cloned via a translation-start (ATG) fusion and with their original stop codons.

1.1 Construction of Plasmid pBAD-Ao-HmaS

A 1131 bp fragment comprising the open reading frame (ORF) for p-hydroxymandelate synthase was amplified by PCR from the chromosomal DNA from Amycolatopsis orientalis NRRL18098 (nucleotides 14957-16030 of accession number AJ223998; amplified region nucleotides 14957-16060) using the following primers:
5′-GTCCACGGTCTCCCATGCAGAATTTCGAGAT-3′ [SEQ ID: No.1] (with Bsa I recognition and cleavage site underlined), and
5′-ACATCCCAAGCTTCACGTTCGAGGTC-3′ [SEQ ID: No.2] (with Hind III cleavage site underlined).

A list of all oligonucleotide sequences used in the context of the present application is presented in Annexe 4 hereto.

Correct size of the amplified fragment was confirmed by agarose gel electrophoresis and the fragment was purified from the gel. The fragment was digested with the enzymes Bsa I and Hind III to generate sticky ends. The plasmid pBAD/Myc-H is C was digested with Nco I and Hind III. The two fragments were subsequently ligated and used for the transformation of chemically competent cells of E. coli Top10 (Invitrogen, Groningen, The Netherlands). The transformants were selected on LB agar plates containing 100 mg/l ampicillin. A plasmid showing the correct insert sequence (as confirmed by sequencing) was given the name pBAD-Ao-HmaS, which has been deposited under the Budapest Treaty at the Deutsche Sammlung for Mikroorganismen und Zellkulturen, Braunschweig, Germany (DSMZ), on Oct. 23^{rd}, 2000, under deposit number DSM 13786, and was used for further investigations.

1.2 Construction of Plasmid PBAD-Ao-HmaO

A 1096 bp fragment comprising the ORF for the p-hydroxymandelate oxidase was amplified by PCR from Amycolatopsis orientalis NRRL18098 chromosomal DNA (nucleotides 16027-17100 of accession number AJ223998; amplified region nucleotides 16027-17101) using the following primers: 5′-CGCTCGGTCATGACGTACGTTTCCCTG-3′ [SEQ ID: No.3] (with BspH I cleavage site underlined), and 5′-ACGAAGAAGCTTATCAAACAACCCCCAG-3′ [SEQ ID: No.4] (with Hind III cleavage site underlined).

Correct size of the amplified fragment was confirmed by agarose gel electrophoresis and the fragment was purified from the gel.

The fragment was digested with the enzymes BspH I and Hind III and the plasmid pBAD/Myc-H is C was digested with Nco I and Hind III. The two fragments were ligated and introduced into E. coli Top10 cells. The transformants were selected on LB agar plates containing 100 mg/l ampicillin. A plasmid showing the correct insert sequence (as confirmed by sequencing) was called pBAD-Ao-HmaO which has been deposited under the Budapest Treaty at the DSMZ on Oct. 23^{rd}, 2000, under deposit number DSM 13791, and was used for further investigations.

1.3 Construction of Plasmid PBAD-Sc-HmaS

A 1091 bp fragment comprising the ORF for the p-hydroxymandelate synthase was amplified by PCR from Streptomyces coelicolor A3(2) strain M145 chromosomal DNA (encoded by nucleotides 1418-2533 of accession AL035640; amplified region nucleotides 1415-2494) using the following primers:
5′-ATGCCGCCCAGTGACATCGCGTACGC-3′ [SEQ ID: No.5] and 5′-CCCTCGGTACCAGGTCATCGGCCGGCCACTTCC-3′ [SEQ ID: No.6] (with Kpn I restriction site underlined).

Correct size of the amplified fragment was confirmed by agarose gel electrophoresis and the fragment was purified from the gel. The amplified fragment was cloned into the Nco I/Kpn I site of the vector pBAD/Myc-H is C as described by Dietmaier et al. (1993, Nucleic Acids Res. 21, 3603-3604).

The resulting plasmid was introduced into E. coli Top10 cells by electroporation. The transformants were selected on LB agar plates containing 100 mg/l carbenicillin.

All colonies resulting from the transformation were collected and the resulting cell suspension was used for plasmid DNA isolation. The total plasmid DNA was digested with Hind III and separated on an agarose gel. A 5 kb DNA fragment being the target plasmid in linear form was isolated from the gel, re-ligated and introduced into chemically competent E. coli Top10 cells by electroporation.

The transformants were selected on LB agar plates containing 100 mg/l carbenicillin.

A plasmid showing the correct insert sequence (as confirmed by sequencing) was given the name pBAD-Sc-HmaS which has been deposited under the Budapest Treaty at the DSMZ on Oct. 23^{rd}, 2000, under deposit number DSM 13790, and was used for further investigations.

1.4 Construction of Plasmid pBAD-Sc-HmaO

A 1149 bp fragment comprising the ORF for the p-hydroxymandelate oxidase was amplified by PCR from the Streptomyces coelicolor A3(2) strain M145 chromosomal DNA (nucleotides 135-1268 of accession AL035640; amplified region nucleotides 132-1267) using the following primers:
5′-ATGCGGGAGCCGCTCACGCTCGAC-3′ [SEQ ID: No.7] and
5′-CCAACTGGTACCTGGTCATCCGTGGCTCCTGTCTCG-3′ [SEQ ID: No.8] (with Kpn I restriction site underlined).

Correct size of the amplified fragment was confirmed by agarose gel electrophoresis and the fragment was purified from the gel. The amplified fragment was cloned into the Nco I/Kpn I site of vector pBAD/Myc-H is C as described by Dietmaier et al. (1993, Nucleic Acids Res. 21, 3603-3604) and the resulting plasmid was subsequently introduced into E. coli Top10 cells. Transformants were selected on LB agar plates containing 100 mg/l carbenicillin.

Plasmid DNA was isolated from all transformants, digested with Hind III and separated on an agarose gel. A 5.2 kb DNA fragment being the target plasmid in linear form was isolated from the gel, relegated and introduced into chemically competent E. coli Top10 cells. The transformants were selected on LB agar plates containing 100 mg/l carbenicillin.

A plasmid showing the correct insert sequence (as confirmed by sequencing) was given the name pBAD-Sc-HmaO, which has been deposited under the Budapest Treaty at the DSMZ on Oct. 23^{rd}, 2000, under deposit number DSM 13789, and was used for further investigations.

### Example 2

### Expression of p-hydroxymandelate Synthase and p-hydroxymandelate Oxidase from A. orientalis and S. coelicolor

Single colonies of the E. coli Top10 strains harboring the plasmids pBAD-Ao-HmaS or pBAD-Sc-HmaS (for p-hydroxymandelate synthase) and the plasmids pBAD-Ao-HmaO or pBAD-Sc-HmaO (for p-hydroxymandelate oxidase) were cultivated in 50 ml LB medium containing 100 mg/l carbenicillin at 30° C. At OD₆₂₀ₙₘ 1.2, the cells were induced by the addition of 0.002% (final concentration) arabinose. After 3.5 hours the cells were harvested and washed with 1 mM MgSO₄ at pH 7.4. Aliquots of washed cells were frozen at −20° C. for later use. As a control, E. coli Top10 harboring plasmid pBAD/Myc-HisC was treated accordingly.

Crude extracts were prepared by sonification in 200 mM potassium phosphate buffer pH 7.5 immediately before use.

### Example 3

### Analysis of p-hydroxymandelate Synthase from A. orientalis and S. coelicolor

3.1 Activity Towards p-hydroxyphenylpyruvate

The assay mixture of 3 ml contained 200 mM potassium phosphate buffer pH 7.5, 5 mM p-hydroxyphenylpyruvate, 10% ethanol (50 mM p-hydroxyphenylpyruvate stock solution in 96% ethanol was used), 44 mM ascorbate, 0.3 mM FeSO₄, and cell free extract leading to a final concentration of 0.6 mg/ml of soluble protein. Boiled extracts were used in control experiments.

The assay was started by the addition of HPP and stopped after 1 hour at 28° C. by the addition of 0.1 ml 1 N HCl to an aliquot of 0.5 ml of the reaction system. The samples were analyzed by HPLC and detected at 215 nm. A Nucleosil-120-5-C18 column (250×4 mm, Macherey-Nagel, Düren, Germany) was used. The column was eluted with eluent A (50 mM H₃PO₄) and eluent B (100% methanol). Gradient: 0-5 min, 0% B; 5-37 min, 0% to 90% B; 3742 min, 90% B; 42-50 min, 90% to 0% B; 50-55 min, 0% B. The flow was 1.0 ml/min, the column temperature was set at 30° C.

65 mg/l p-hydroxymandelate was produced within 1 hour with cell free extracts derived from E. coli/pBAD-Ao-HmaS, while 35.6 mg/l p-hydroxymandelate was produced with extracts derived from E. coli/pBAD-Sc-HmaS. No p-hydroxymandelate was produced in the control experiments (crude extracts of E. coli/pBAD/Myc-HisC and boiled extract).

3.2 Activity Towards Phenylpyruvate

The assay mixture of 3 ml contained 200 mM potassium phosphate buffer pH 7.5, 5 mM phenylpyruvate, 44 mM ascorbate and 0.3 mM FeSO₄ and cell free extract at a final concentration of 0.6 mg/ml of soluble protein.

The assay was started at 28° C. by the addition of the cell free extract and stopped by the addition of 0.1 ml 1 N HCl to an aliquot of 0.5 ml of the reaction system. Samples were analyzed by HPLC as described above. Both boiled extracts and the crude extract of E. coli/pBAD/Myc-HisC were used in control experiments.

63 mg/l mandelate was produced within 1 hour using cell free extract derived from E. coli/pBAD-Ao-HmaS. Cell free extract derived from E. coli/pBAD-Sc-HmaS produced 25 mg/l mandelate within 6 hours. No mandelate was detected in the control experiments.

### Example 4

### Analysis of Phydroxymandelate Oxidase from A. orientalis and S. coelicolor

4.1 Activity Towards D,L-p-hydroxymandelate

100 μl of the crude extract containing 0.5-0.8 mg protein was incubated with 100 mM potassium phosphate buffer pH 7.5, 2 mM D,L-p-hydroxymandelate, 20 mg/l catalase in a total volume of 1 ml. The oxidation of phydroxymandelate was monitored spectrophotometrically at 340 nm. To correct for non-specific oxidation of p-hydroxymandelate, control assays were run using an assay mixture without cell free extract.

The specific activity was 30 nmol*min⁻¹*mg⁻¹ total protein for the extracts of E. coli Top10/pBAD-Sc-HmaO and 5 nmol*min⁻¹*mg⁻¹ total protein for E. coli Top10/pBAD-Ao-HmaO.

4.2 Activity Towards (S)- or (R)-mandelate

The assay mixture of 3 ml contained 100 mM potassium phosphate buffer pH 7.5, 2 mM (S)- or (R)-mandelate, 20 mg/l catalase and cell free extract at a final concentration of 0.6 mg/ml of soluble protein.

The assay was started by addition of the cell free extract and stopped by the addition of 0.1 ml 1 N HCl to an aliquot of 0.5 ml of the reaction system. Samples were analyzed by HPLC by the method of Example 3. Boiled extracts were used in control experiments.

No phenylglyoxylate was produced with cell free extracts derived from E. coli/pBAD-Ao-HmaO or E. coli/pBAD-Sc-HmaO using (R)-mandelate as a substrate. With (S)-Mandelate as a substrate 9 mg/l phenylglyoxylate were produced within 1 hour using cell free extract from E. coli/pBAD-Ao-HmaO, and 27 mg/ml phenylglyoxylate was produced within 1 hour with cell free extract derived from E. coli/pBAD-Sc-HmaO.

### Example 5

### Construction of Plasmids pMAL-Nu-HmaS and pMAL-Nu-HmaO

Nocardia uniformis subsp. tsuyamanensis ATCC 21806 was obtained from the American Type Culture Collection (Manassas, Va., USA). N. uniformis was cultivated and genomic DNA was isolated as described in J. Biol. Chem. 1998, 273, 30695-30703. Genes were cloned into the pMAL-c2 expression vector (New England BioLabs, Beverly, Mass., USA) with their original stop codons resulting in a maltose-binding protein fusion protein. HmaS was also cloned into pET-29b (Novagen, Madison, Wis., USA) as a C-terminal His-6 tagged fusion.

5.1 Construction of Plasmid pMAL-Nu-HmaS

A 1052 bp fragment comprising the open reading frame (ORF) for phydroxymandelate synthase was amplified by PCR from Nocardia uniformis subsp. tsuyamanensis chromosomal DNA (nucleotides 52-1086 of SEQ. ID: No.9, encoding the protein of SEQ.ID: No.10, given in annexe 1; amplified region nucleotides 55-1089) using the following primers:
5′-AGAATTCGCGGCACAGGCAGGCAGCG-3′ [SEQ ID: No.11] (with EcoR I cleavage site underlined) and
5′-TTATAAGCTTTCAGCGCTCGGTCCGGTGGC-3′ [SEQ ID: No.12] (with Hind III cleavage site underlined).

Correct size of the amplified fragment was confirmed by agarose gel electrophoresis and the fragment was purified from the gel. The fragment was digested with EcoR I and Hind III. The plasmid pMAL-c2 was digested with EcoR I and Hind III and the two fragments were subsequently ligated and transformed by electroporation into E. coli TB1 cells (New England BioLabs, Beverly, Mass., USA). The transformants were selected on LB agar plates containing 100 mg/l ampicillin. A plasmid showing the correct insert sequence (as confirmed by sequencing) was given the name pMAL-Nu-HmaS, which has been deposited under the Budapest Treaty at the ATCC on Oct. 27^{th}, 2000, under Patent Deposit Designation PTA-2639, and used for further investigations.

5.2 Construction of Plasmid pET-Nu-HmaS

A 1051 bp fragment comprising the open reading frame (ORF) for phydroxymandelate synthase was amplified by PCR from Nocardia uniformis subsp. tsuyamanensis chromosomal DNA (nucleotides 52-1086 of SEQ. ID: No.9, encoding the protein of SEQ.ID: No.10, given in annexe 1; amplified region nucleotides 52-1086) using the following primers:
5′-TATACCATGGCGGCACAGGCAGGC-3′ [SEQ ID: No.13](with Nco I cleavage site underlined) and
5′-TTATAAGCTTGCGCTCGGTCCGGTGGC-3′ [SEQ ID: No.14] (with Hind III cleavage site underlined).

Correct size of the amplified fragment was confirmed by agarose gel electrophoresis and the fragment was purified from the gel. The fragment was digested with Nco I and Hind III. The plasmid pET-29b was digested with Nco I and Hind III and the two fragments were subsequently ligated and transformed by electroporation into E. coli BL21 (DE3) cells (Novagen, Madison, Wis., USA). The transformants were selected on LB agar plates containing 50 mg/l kanamycin. A plasmid showing the correct insert sequence (as confirmed by sequencing) was given the name pET-Nu-HmaS, which has been deposited under the Budapest Treaty at the ATCC on Oct. 27^{th}, 2000, under Patent Deposit Designation PTA-2638, and used for further investigations.

5.3 Construction of Plasmid pMAL-Nu-HmaO

A 1144 bp fragment comprising the ORF for the p-hydroxymandelate oxidase was amplified by PCR from Nocardia uniformis chromosomal DNA (nucleotides 50-1177 of SEQ. ID: No.15, encoding the protein of SEQ.ID: No.16, given in annexe 2; amplified region nucleotides 53-1180) using the following primers:
5′-AGAATTCGGCGTCCGCAACTCCGCAG-3′ [SEQ ID: No.17] (with EcoR I cleavage site underlined) and
5′AATAAGCTTTCAGGGCGCACCTCGCC-3′ [SEQ ID: No.18] (with Hind III cleavage site underlined).

Correct size of the amplified fragment was confirmed by agarose gel electrophoresis and the fragment purified from the gel. The fragment and plasmid pMAL-c2 were digested with EcoR I and Hind III. The two fragments were ligated and transformed into E. coli TB1 cells by electroporation. The transformants were selected on LB agar containing 100 mg/l ampicillin. A plasmid showing the correct insert sequence (as confirmed by sequencing) was named pMAL-Nu-HmaO, deposited under the Budapest Treaty at the ATCC on Oct. 27^{th}, 2000, under Patent Deposit Designation PTA-2637, and used for further investigations.

### Example 6

### Preparation of p-hydroxymandelate Synthase and p-hydroxymandelate oxidase from N. uniformis

6.1 Expression of p-hydroxymandelate Synthase (First Method) and of p-hydroxymandelate Oxidase

Single colonies of the E. coli TB1 strains harboring the plasmids pMAL-Nu-HmaS or pMAL-Nu-HmaO were cultivated in 100 ml LB medium (+0.02% glucose) containing 100 mg/l ampicillin at 37° C. At OD₆₀₀ₙₘ 0.6, the cells were induced by the addition of 0.3 mM (final concentration) isopropylthiogalactoside (IPTG). After 3 hours, the cells were harvested, resuspended in buffer (20 mM Tris-HCl pH 7.4, 200 mM NaCl, 1 mM EDTA for HmaO and 20 mM Tris-HCl pH 7.5, 10 mM EDTA, 1% Triton X-100 for HmaS) and frozen at −20° C. for later use. As a control E. coli TB1 cells containing the plasmid pMAL-c2 were treated identically.

6.2 Purification of p-hydroxymandelate Synthase (First Method) and of p-hydroxymandelate Oxidase

Crude extracts of resuspended pellets were prepared by sonication immediately prior to use. Insoluble cell debris was removed from the extract by centrifugation and the resulting cell free extract applied to an amylose resin column. The desired protein was obtained in ≧80% purity by elution with buffer (20 mM Tris-HCl pH 7.4, 200 mM NaCl, 1 mM EDTA) containing 10 mM maltose. The partially purified extract was used in further investigations.

6.3 Expression of p-hydroxymandelate Synthase (Second Method)

Single colonies of the E. coli BL21 (DE3) strains harboring the plasmids pET-Nu-HmaS were cultivated in 100 ml LB medium containing 50 mg/l kanamycin at 37° C. At OD₆₀₀ₙₘ 0.6, the cells were induced by the addition of 1 mM (final concentration) IPTG. After 4 hours, the cells were harvested, resuspended in buffer (50 mM sodium phosphate pH 8, 300 mM NaCl, 10 mM imidazole) and frozen at −20° C. for later use.

6.4 Purification of p-hydroxymandelate Synthase (Second Method)

A crude extract was prepared by sonication immediately prior to use. Insoluble cell debris was removed from the extract by centrifugation and the resulting cell free extract was batch incubated with Ni-NTA resin (Qiagen, Valencia, Calif., USA) for 1 hour at 200 rpm. The CFE-resin slurry was poured into a column and the desired protein was obtained in ≧90% purity by elution with buffer (50 mM sodium phosphate pH 8, 300 mM NaCl) containing increasing concentrations of imidazole. The partially purified extract was used in further investigations.

### Example 7

### Activity of p-hydroxymandelate Synthase from N. uniformis

The assay mixture of 0.4 ml contained 50 mM Tris-HCl buffer pH 8.0, 5 mM p-hydroxyphenylpyruvate, 0.7% ethanol (50 mg/ml p-hydroxyphenylpyruvate stock solution was used), 0.5 mM dithiothreitol (DTT), 0.1 mM ascorbate, 0.025 mM FeSO₄, and partially purified HmaS. The assay was started by addition of HmaS and stopped after 1 hour at 30° C. by heat inactivation at 100° C., 5 min. Denatured protein was removed by centrifugation and 0.35 ml of the reaction system used in a coupled assay with HmaO. The assay was started with addition of 0.1 mg/ml HmaO and an increase in absorbance at 332 nm was observed due to the oxidaton of phydroxymandelate to HPGL. No HPGL was produced in control experiments.

### Example 8

### Activity of p-hydroxymandelate Oxidase from N. uniformis

The assay mixture of 0.5 ml contained 50 mM Tris-HCl buffer pH 8 and 2 mM (R,S)-p-hydroxymandelic acid. The assay was initiated with the addition of partially purified HmaO at a final concentration of 0.1 mg/ml. Formation of p-hydroxyphenylglyoxylate (HPGL) was monitored spectrophotometrically at 332 nm. No formation of HPGL was observed in the absence of substrate. The specific activity for HmaO was determined to be 240 μmol*min⁻¹*mg⁻¹.

In separate experiments with wild-type enzyme purified from N. uniformis, the oxidase was shown to be flavin-dependent and specific for (S)-mandelic acid.

### Example 9

### Construction of Plasmids pGEM-mdlB and DGEM-Bldm

Pseudomonas putida ATCC 12633 was obtained from the American Type Culture Collection (Manassas, Va., USA). P. putida was cultivated in LB medium (10 g/l Tryptone Peptone (Difco), 5 g/l yeast extract (Difco), 5 g/NaCl) at 28° C. The genomic DNA from this P. putida strain was isolated after overnight cultivation using the standard protocol described by Ausubel et al. (1990, Current Protocols in Molecular Biology, chapter 2.4.3, step 1 to 9, Whiley-Interscience, New York). The crude chromosomal DNA was treated with RNAse (20 mg/[), and subsequently with phenol/chloroformlisoamyl alcohol (25:24:1) to remove proteins.

A 1409 bp fragment comprising the ORF for the (S)-mandelate dehydrogenase was amplified by PCR from the chromosomal DNA from Pseudomonas putida ATCC 12633 (encoded by nucleotides 2251-3432 of accession J05293; amplified region nucleotides 2110-3518) using Taq DNA-polymerase and the following primers:
5′-ACTCGCCAAGGGCTATGGTGTCC-3′ [SEQ ID: No.19] and
5′-GCCAACAGTTCCAACAGCGGTGTG-3′. [SEQ ID: No.20]

Correct size of the amplified fragment was confirmed by agarose gel electrophoresis.

The amplified fragment was cloned in vector pGEM-T (Promega, Madison, Wis., USA) and E. coli XL1-Blue MRF′ (Stratagene, La Jolla, Calif., USA) was transformed therewith. The transformants were selected on LB agar plates containing 100 mg/l ampicillin.

Sequencing of the insert of four different clones revealed that all clones had GC instead of CG at position 2291/2292. One of the clones, without additional mutations, and named pGEM-Bldm, has been deposited under the Budapest Treaty at the DSMZ on Oct. 23^{rd}, 2000, under deposit number DSM 13787. This original deposit, which, wrongfully, has been made under the name pGEM-mdlB but correctly should have been named pGEM-Bldm, was used in further cloning experiments. Another clone, with an additional silent mutation, named pGEM-mdlB, was used to demonstrate the desired activity. It is noted, that in said latter plasmid the orientation of the mandelate dehydrogenase gene matches the orientation of the vector-bome lac promotor.

### Example 10

### Expression of (S)-mandelate Dehydrogenase from P. putida

E. coli XL1-MRF′/pGEM-mdlB was cultured in LB medium containing 100 mg/l carbenicillin and 1 mM IPTG. After 16 hours of cultivation at 37° C., cells were harvested by centrifugation and re-suspended in 50 mM potassium phosphate buffer, pH 6.8. Cells were disintegrated by ultrasonic treatment and non-broken cells and cell fragments were removed by centrifugation (5.000 g, 10 min, 4° C.). Finally, membranes were collected by an ultra centrifugation step (1 hour, 200.000 g, at 4° C.) and then re-suspended in 4 ml of 50 mM potassium phosphate buffer, pH 6.8 and kept on ice.

The protein concentration of the membrane fraction was determined by a modified Lowry protein assay (Sandermann and Strominger, 1972, J. Biol. Chem. 247: 5123-5131), in which 1% of SDS is added to the Lowry reagent A. The membrane fraction contained about 1 mg/ml protein.

### Example 11

### Proof of Activity of the (S)-mandelate Dehydrogenase

The (S)-mandelate dehydrogenase activity was spectrophotometrically determined using 2,6-dichlorophenol-indophenol as acceptor dye by the method of Hegeman (1966, J. Bacteriol. 91: 1140-1154). Stereoselectivity of (S)-mandelate dehydrogenase was examined by using (S)- or (R)-mandelate as a substrate. Further it was analyzed, whether the (S)-mandelate dehydrogenase can also take p-hydroxymandelate as a substrate.

Membrane fractions of E. coli XL1 MRF′/pGEM-mdlB led to a detectible change in absorbance at 600 nm within 5 minutes with (S)-mandelate and with (R, S)-p-hydroxymandelate as a substrate. With (R)-mandelate, no changes in absorbance were observed.

### Example 12

### Isolation of the D-p-hydroxyphenylglycine aminotransferase (HpgAT) gene

The D-p-hydroxyphenylglycine aminotransferase gene was isolated from Pseudomonas putida NCIMB 12565 (National Collection of Industrial and Marine Bacteria, Aberdeen, Scotland, UK). DNA was extracted from exponentially growing cells (OD₆₂₀ₙₘ 1.9) using the standard protocol described by Ausubel et al. (1990, Current Protocols in Molecular Biology, chapter 2.4.3, step 1 to 9, Whiley-Interscience, New York). The crude chromosomal DNA was treated with RNAse (20 mg/l), and subsequently with phenol/chloroform/isoamyl alcohol (25:24:1) to remove proteins. The chromosomal DNA was then partially digested with Sau3A I. The digested DNA was run on a 0.6% agarose gel and DNA fragments between 4 and 10 kb in size were isolated.

Vector DNA was prepared by the digestion of 1 μg of pZErO-2 (Invitrogen, Groningen, The Netherlands) with BamH I according to the protocol of Invitrogen.

Vector DNA and P. putida chromosomal DNA fragments were ligated with T4 DNA ligase. The ligation mixture was used to transform chemically competent E. coli Top10 cells. Transformants were plated onto LB medium with 50 mg/l kanamycin. In total 5000 colonies were obtained which formed the primary gene library. All 5000 colonies were pooled in LB medium supplemented with 50 mg/l kanamycin. After addition of glycerol to a final concentration of 15%, the primary gene bank was stored in aliquots of 1 ml at −80° C.

Cultures of 1800 colonies were prepared in 150 μl LB medium supplemented with 50 mg/4 kanamycin in microtiter plates. The cultures were cultured overnight at 28° C. and harvested by centrifugation in an Eppendorf 5804 R centrifuge (Eppendorf, Hamburg, Germany). The cells were washed with 50 mM KPO₄ buffer, pH 7.0, and re-suspended in 180 μl reaction mix (100 mM potassium phosphate, pH 7.0, 15 mM α-ketoglutarate, 0.1 mM pyridoxal-phosphate and 0.5% v/v Triton X-100).

The reaction was started by adding D-p-hydroxyphenylglycine to a final concentration of 5 mM. The OD₃₄₀ₙₘ in each well was monitored during 20 minutes using an Optimax microtiter plate reader (Molecular Devices, Sunnyvale, Calif., USA). A negative control (non transformed E. coli Top10) and a positive control (P. putida NCIMB 12565) were treated accordingly.

Of 1800 clones screened, one showed significant increase in the OD₃₄₀ₙₘ relative to the negative control due to the formation of HPGL. This clone contained the P. putida D-HpgAT gene on a 12 kb plasmid, pZErOTagp. Sequencing of pZErOTagp revealed the complete nucleotide sequence of the D-HpgAT gene. The sequence of this gene is listed as nucleotides 51 to 1376 of [SEQ ID: No.21, encoding the protein of SEQ.ID: No.22], as shown in annexe 3.

### Example 13

### Construction of Plasmid pBAD-HpgAT

The P. putida D-p-hydroxyphenylglycine aminotransferase gene was subcloned into pBAD/Myc-H is C using PCR. The HpgAT ORF was amplified using
5′-GTGCACGGTCTCGCATGTCTATTTATAGCGATTATGAACGTAAMC-3′ [SEQ ID: No.23] and
5′-GTGCACGGTCTCCTCGAGTTAGCCCAGGAGGTTTTCTTCAGC-3′ [SEQ ID: No.24] as primers (with Bsa I recognition and cleavage site underlined), and chromosomal DNA of Pseudomonas putida NCIMB 12565 as template. Correct size (1361 bp) of the amplified fragment was confirmed by agarose gel electrophoresis.

The fragment was digested with Bsa 1. Vector pBAD/Myc-H is C was digested with Xho I and Nco 1. The purified digested pBAD/Myc-H is C vector and the digested insert DNA were ligated with T4 DNA ligase.

E. coli Top10 cells were transformed by electroporation with the recombinant plasmid and plated on LB medium supplemented with 100 mg/l carbenicillin.

Colony-pCR was performed and eight PCR positives as well as E. coli Top10/pBAD/Myc-H is C (as negative control) were cultured in LB medium supplemented with 100 mg/l carbenicillin with and without 0.002% arabinose as an inducer. The cells were harvested and tested for HpgAT activity as described in Example 12. All 8 colonies showed HpgAT activity with arabinose as inducer. One of these colonies, carrying a plasmid showing the correct insert sequence (as confirmed by sequencing), and named E. coli pBAD-HpgAT, was deposited under the Budapest Treaty at the Deutsche Sammlung für Mikroorganismen und Zellkulturen, Braunschweig, Germany (DSMZ), on Oct. 23^{rd}, 2000, deposit number DSM 13788.

### Example 14

### Preparation of Cell Free Extracts

E. coli Top10/pBAD-HpgAT was cultured in LB medium supplemented with arabinose (0.002%) and carbenicillin (100 mg/l) overnight at 28° C. The cells were harvested by centrifugation and cell free extracts were prepared from a suspension (1 g of cells plus 7 ml of 50 mM KPO₄, pH 7.0) by sonification and subsequent centrifugation.

### Example 15.a

### Activity of the D-HpgAT Towards p-hydroxyphenylglyoxylate

3 ml reaction mixture containing 100 mM potassium phosphate buffer pH 7.0, 60 mM L-glutamate, 0.1 mM pyridoxal phosphate and cell free extract (0.27 mg protein, example 14) was incubated in a cuvette at 20° C. This mixture was used as blank in the spectrophotometric activity measurement at 340 nm. The reaction was started by the addition of p-hydroxyphenylglyoxylate (0.67 mM final concentration) into the reaction mixture. The maximum specific activity in this reaction was 110 nmole per minute per mg protein at 20° C.

### Example 15.b

### Activity of the D-HpgAT Towards D-pG and D-HPG

4 ml reaction mixture containing 100 mM potassium phosphate buffer pH 8.0, 15 mM α-ketoglutarate, 0.1 mM pyridoxal phosphate, and cell free extract (0.01 mg protein, example 14) was incubated at 30° C. The assay was started by the addition of the substrate D-pG (4 mM). At certain time intervals, aliquots of 1 ml were taken and transferred to 0.4 ml 1 M H₃PO₄ to stop the reaction. Samples were analyzed by HPLC (Astec Chirobiotic T 250 mm×4.6 mm 5 μm column, Advanced Separation Technologies, Whippany, N.J., 20 μl injection volume, column temperature 22° C., 1.0 ml/min of 80% 15 mM ammonium acetate pH 4.1 and 20% methanol, detection at 215 nm). Within 20 min, 7% of D-pG has been converted to phenylglyoxylate, while in a similar experiment carried out with D-HPG as a substrate, 12% of D-HPG has been converted to p-hydroxyphenylglyoxylate.

### Example 16

### pH Optimum of the D-HpgAT

pH optimum of the enzyme was determined in 5 ml reaction mixtures containing 100 mM buffer of a certain pH, 6.5 mM p-hydroxyphenylglyoxylate, 0.05 mM pyridoxal phosphate and 100 μl cell free extract (1.35 mg protein, example 14). The reaction was started by adding 300 μmole L-glutamate (at the appropriate pH, 60 mM final concentration). After certain time intervals at 35° C., samples were taken and stopped by the addition of the same volume of 0.2 M H₃PO₄. The samples were analyzed by HPLC (Biorad HPX-87C 300 mm×7.8 mm, 20 μl injection volume, column temperature 80° C., 1 m/min 5 mM calcium phosphate) and detected at 210 nm.

The enzymatic activity was tested in the pH range 5 to 11. KH₂PO₄/Na₂HPO₄ buffer (pH 5 to 8), TRIS-HCl buffer (pH 8 to 9), CHES-NaOH buffer (pH 9 to 10) and CAPS-NaOH buffer (pH 10 to 11) were applied, respectively.

The pH optimum of the enzyme was found to be between pH 8.5 and 9. Significant activity was found between pH 6 and pH 7.5. This is in contrast to Wiyakrutta and Meevootisom (1997, J. Biotechnol., 55: 193-203), who could not observe any activity below pH 7.5 of a D-HpgAT from Pseudomonas stutzeri.

### Example 17

### Pyruvate as an Amino Acceptor for the D-HpgAT

The spectrophotometric assay of Example 12 (however, without Triton in the assay mixture) was applied with pyruvate instead of α-ketoglutarate as the amino acceptor. An increase in absorbance could be observed due to the formation of HPGL. Hence, P. putida D-HpgAT is able to use pyruvate as amino acceptor. The aminotransferase of P. stutzeri, described by Wiyakrutta and Meevootisom (1997, J. Biotechnol., 55: 193-203), used α-ketoglutarate as the sole amino acceptor.

### Example 18

### Enantioselectivity of the D-HpgAT (I)

The spectrophotometric assay of Example 12 was used to test the activity of D-HpgAT towards L-HPG. No increase in absorption was observed when L-HPG was used as substrate. After adding D-HPG, the absorption started to increase. Hence, the present D-HpgAT is selective for D-p-hydroxyphenylglycine.

To test the enantioselectivity for L-glutamate, a 3 ml reaction mixture containing 100 mM potassium phosphate buffer pH 7.0, 60 mM D-glutamate and 0.1 mM pyridoxal phosphate was incubated at 20° C. This mixture was used as blank in the spectrophotometric activity measurement at 340 nm. p-Hydroxyphenylglyoxylate (0.5 mM final concentration) was added to the reaction mixture. After 2 minutes of incubation, the reaction was started by the addition of 20 μl cell free extract (0.27 mg protein, example 14) to the reaction mixture. No decrease in absorption could be identified when D-glutamate was used as substrate. After adding L-glutamate, the absorption started to decrease. Thus, the current D-HpgAT is selective for L-glutamate.

### Example 19

### Enantioselectivity of the D-HpgAT (II)

For further proof of the strict enantioselectivity, 2 reaction mixtures (50 ml final volume) containing 100 mM potassium phosphate buffer pH 8.0, 13 mM p-hydroxyphenylglyoxylate, 0.05 mM pyridoxal phosphate and cell free extract (13.5 or 1.35 mg protein, example 14), were pre-incubated at 35° C. for 10 minutes. The reaction was started by adding 34.8 mmole L-glutamate, pH 8.0 (final concentration 0.7 M).

Samples (1 ml) were taken after regular intervals, stopped by the addition of 2 ml of 0.2 M H₃PO₄ and analyzed by HPLC by the method of example 15. Only D-HPG (not L-HPG) was produced in the reaction.

### EXPERIMENTAL PART II (EXAMPLES 20-35)

### Construction and Testing of Artificial D-(H)PG Biosynthesis Operons

General Considerations for Examples 20-35

As expression vector for the artificial D-(H)PG biosynthesis operon, plasmid pJF119EH was chosen. This broad host range vector, constructed by Furste, et al. (1986, Gene, 48:119-131), is suitable for protein expression in a variety of gram negative bacteria. The pJF119EH expression system uses the IPTG inducible tac promoter and carries the lac repressor (lac I^{q} gene), which keeps the expression of the cloned foreign gene in the absence of the inducer extremely low.

In all cases the different genes belonging to the D-(H)PG operon were amplified by PCR from the appropriate plasmids described in example 1 example 9, and example 13. To ensure the presence of an optimal ribosomal binding site (RBS) in front of those genes, the RBS as it is present in pBAD/Myc-H is C was included.

### Example 20

### Construction of Plasmid pJF-Sc-HmaS

The S. coelicolor p-hydroxymandelate synthase gene was subcloned in pJF 119EH using PCR. The HmaS ORF including RBS was amplified using
5′-GGGAATTCAGGAGGAATTAACCATGCCGCCgAGcGAC-3′ [SEQ ID: No.25] (with EcoRI restriction site underlined, start codon double underlined, and change of codon 3 and 4, indicated by non capital letters, to more frequently used ones in E. coli), and
5′-GAATTCCCATATTCTAGAAGGTCATCGGCCGGCCACT-3′ [SEQ ID: No.26] (with Xba I restriction site underlined, and stop codon double underlined) as primers, and pBAD-Sc-HmaS (see example 1.3) plasmid DNA as template. Correct size (1120 bp) of the amplified fragment was confirmed by agarose gel electrophoresis.

The fragment and the plasmid pJF119EH were digested with EcoRI and Xba 1. The two fragments were ligated and transformed in E. coli DH5α. Transformants were selected on LB agar plates containing 100 mg/l ampicillin. A plasmid showing the correct insert sequence (as confirmed by sequencing) was called pJF-Sc-HmaS.

### Example 21

### Construction of Plasmid pJF-Ao-HmaS

The A. orientalis p-hydroxymandelate synthase gene was subcloned in pJF119EH using PCR. The HmaS ORF including RBS was amplified using
5′-TGGGAATTCAGGAGGAATTAACCATGCAG-3′ [SEQ ID: No.27] (with EcoRI restriction site underlined, and start codon double underlined), and
5′-CGGCCAGGTCTAGATACGTCATCGCCG-3′ [SEQ ID: No.28] (with Xba I restriction site underlined, and stop codon double underlined) as primers, and pBAD-Ao-HmaS (see example 1.1) plasmid DNA as template. Correct size (1115 bp) of the amplified fragment was confirmed by agarose gel electrophoresis.

The fragment and the plasmid pJF119EH were digested with EcoRI and Xba 1. The two fragments were ligated and transformed in E. coli DH5α. Transformants were selected on LB agar plates containing 100 mg/l ampicillin. A plasmid showing the correct insert sequence (as confirmed by sequencing) was called pJF-Ao-HmaS.

### Example 22

### Construction of Plasmid pJF-Sc-HmaO

The S. coelicolor p-hydroxymandelate oxidase gene was subcloned in pJF119EH using PCR. The HmaO ORF including RBS was amplified using
5′-TGGGTCTAGAGGAGGAATTAACCATGCGcGAGCCG-3′ [SEQ ID: No.29] (with XbaI restriction site underlined, start codon double underlined and change of codon 2, indicated by non capital letters, to a more frequently used one in E. coli) and
5′-GAATTCCCATAGCATGCCTGGAAATCCGTGGCTCC-3′ [SEQ ID: No.30] (with Sph I restriction site underlined, and stop codon double underlined) as primers and pBAD-Sc-HmaO (see example 1.4) plasmid DNA as template. Correct size (1178 bp) of the amplified fragment was confirmed by agarose gel electrophoresis.

The fragment and the plasmid pJF119EH were digested with Xba I and Sph 1. The two fragments were ligated and transformed in E. coli DH5α. Transformants were selected on LB agar plates containing 100 mg/l ampicillin. A plasmid showing the correct insert sequence (as confirmed by sequencing) was called pJF-Sc-HmaO and used for further investigation.

### Example 23

### Construction of Plasmid pJF-Sc-HmaS-Sc-HmaO

In this example, the cloned Sc-HmaS and Sc-HmaO from examples 20 and 22 were combined in the expression vector pJF119EH. The Sc-HmaO gene in pJF-Sc-HmaO was excised from the expression vector by digestion with Xba I and Sph 1, and this DNA fragment containing the Sc-HmaO gene was purified by gel electrophoresis. Plasmid pJF-Sc-HmaS was digested with Xba I and Sph 1, ligated together with the Sc-HmaO Xba I/Sph I fragment and transformed in E. coli DH5α. Transformants were selected on LB agar plates containing 100 mg/A ampicillin. Different transformants were selected, plasmid minipreps prepared, and the plasmid DNA analyzed by restriction mapping. A plasmid comprising the two genes in the correct order in pJF119EH was designated pJF-Sc-HmaS-Sc-HmaO and used for further investigation.

### Example 24

### Construction of Plasmid pJF-Ao-HmaS-Sc-HmaO

In this example, the cloned Ao-HmaS and Sc-HmaO from examples 21 and 22 were combined in the expression vector pJF119EH, in the same way as it has been described for the construction of pJF-Sc-HmaS-Sc-HmaO in example 23. A plasmid comprising the two genes in the correct order in pJF119EH was designated pJF-Ao-HmaS-Sc-HmaO and used for further investigation.

### Example 25

### Construction of Plasmid pJF-HpgAT

The P. putida D-p-hydroxyphenylglycine aminotransferase gene was subcloned in pJF119EH using PCR. The HpgAT ORF including RBS was amplified using
5′-TTCCCAAGCTTACAGGAGGAATTAACCATLG-3′ [SEQ ID: No.31] (with Hind III restriction site underlined, start codon double underlined) and
5′-GTACCAGCTGCAAAGCTTGAGTTAGCCCAG-3′ (SEQ ID: No.32] (with Hind III restriction site underlined, and stop codon double underlined) as primers and pBAD-HpgAT (see example 13) plasmid DNA as template. Correct size (1378 bp) of the amplified fragment was confirmed by agarose gel electrophoresis.

The fragment and the plasmid pJF119EH were digested with Hind III. The two fragments were ligated and transformed in E. coli DH5α. Transformants were selected on LB agar plates containing 100 mg/l ampicillin. Insertion of the desired fragment in same direction as the tac promoter was confirmed using restriction analysis. A plasmid showing the correct insert sequence with the exception of one silent mutation (change of codon 46 from GCG to GCA) was called pJF-HpgAT and used for further investigations.

### Example 26

### Construction of Plasmid pJF-Sc-HmaS-Sc-HmaO-HDgAT

In this example, the cloned HpgAT of example 25 was subcloned in plasmid pJF-Sc-HmaS-Sc-HmaO of example 23. The HpgAT gene in pJF-HpgAT was excised from the expression vector by digestion with Hind III, and the DNA fragment containing the HpgAT gene was purified by gel electrophoresis. Plasmid pJF-Sc-HmaS-Sc-HmaO was digested with Hind III. After dephosphorylation, this fragment was ligated together with the HpgAT Hind III fragment and transformed in E. coli DH5α. Transformants were selected on LB agar plates containing 100 mg/l ampicillin. Different transformants were selected, plasmid minipreps prepared, and the plasmid DNA analyzed by restriction mapping. A plasmid comprising the three genes in the correct order and direction in pJF119EH was designated pJF-Sc-HmaS-Sc HmaO-HpgAT and used for further investigation.

### Example 27

### Construction of Plasmid pJF-Ao-HmaS-Sc-HmaO-HpgAT

In this example, the cloned HpgAT of example 25 was subcloned in plasmid pJF-Ao-HmaS-Sc-HmaO of example 24 in the same way as described for the construction of pJF-Sc-HmaS-Sc-HmaO-HpgAT in example 26. A plasmid comprising the three genes in the correct order in pJF119EH was designated pJF-Ao-HmaS-Sc-HmaO-HpgAT and used for further investigation.

### Example 28

### Construction of Plasmid pJF-Sc-HmaS-mdlB-HpgAT

In this example, the cloned Sc-HmaO present in pJF-Sc-HmaS-Sc-HmaO-HpgAT (example 26) was exchanged against the mdlB gene of P. putida resulting in plasmid pJF-Sc-HmaS-mdlB-HpgAT. The P. putida mandelate dehydrogenase gene was amplified by PCR including the RBS sequence as it is present in plasmid pBAD/Myc-H is C using
5′-GGGTCTAGAGGAGGAATTAACCATGACCAGAATCTCTTT-3′ [SEQ ID: No.33] (with Xba I restriction site underlined, and start codon double underlined) and
5′-CTGCAGAACCAGCATGGTGGTCAGTACTTCACTCATGCG-3′ [SEQ ID: No.34] (with Bst XI restriction site underlined, and stop codon double underlined) as primers and pGEM-Bldm (see example 9) plasmid DNA as template. Correct size (1236 bp) of the amplified fragment was confirmed by agarose gel electrophoresis.

The fragment was digested with Xba I and BstX I. Plasmid pJF-Sc-HmaS-Sc-HmaO-HpgAT was digested with Xba I and Sph I leading to a 7692 bp fragment of the plasmid without HmaO gene. The purified 7692 bp Xba I/Sph I fragment was ligated with the mdlB ORF containing Xba I/BstX I fragment and transformed in E. coli DH5α. Transformants were selected on LB agar plates containing 100 mg/l ampicillin. Different transformants were selected, plasmid minipreps prepared, and the plasmid DNA analyzed by restriction mapping. A plasmid comprising the three genes in the correct order in pJF119EH was designated pJF-Sc-HmaS-mdlB-HpgAT and used for further investigation.

### Example 29

### Construction of Plasmid pJF-Ao-HmaS-mdlB-HpgAT

In this example, the cloned Sc-HmaO present in pJF-Ao-HmaS-Sc-HmaO-HpgAT (example 27) was exchanged against the mdlB gene of P. putida resulting in plasmid pJF-Ao-HmaS-mdlB-HpgAT, in the same way as described for the construction of pJF-Sc-HmaS-mdlB-HpgAT in example 28.

A plasmid comprising the three genes in the correct order in pJF119EH was designated pJF-Ao-HmaS-mdlB-HpgAT and used for further investigation.

### Example 30

### Expression of Artificial D-(H)PG Cluster on Plasmids pJF-Sc-HmaS Sc-HmaO-HpgAT, pJF-Ao-HmaS-Sc-HmaO-HpgAT, pJF-Sc-HmaS-mdlB HpgAT, and pJF-Ao-HmaS-mdlB-HpgAT

Single colonies of the E. coli DH5α strains harboring the plasmids pJF-Sc-HmaS-Sc-HmaO-HpgAT, pJF-Ao-HmaS-Sc-HmaO-HpgAT, pJF-Sc-HmaS-mdlB-HpgAT, or pJF-Ao-HmaS-mdlB-HpgAT were used to inoculate 10 ml of LB medium containing Ampicillin (100 μg/ml) and incubated at 30° C. for 16 hours.

1 ml of these cultures were subsequently used to inoculate 50 ml of the same medium. Cells were grown at 30° C. with 180 rpm. At OD₆₂₀ nm 0.8, the cells were induced by the addition of 0.1 mM IPTG. After 4 hours, the cells were harvested and washed with 100 mM potassium phosphate buffer, pH 7.5. Aliquots of washed cells were frozen at −20° C. for later use. As a control E. coli DH5α harboring plasmid pJF119EH was treated accordingly.

Crude extracts were prepared with B-pER™ (in phosphate buffer) (Pierce, Rockford, Ill., USA) immediately before use.

### Example 31

### In Vitro Production of D-HPG from p-hydroxyphenylpyruvate

The assay mixture of 3 ml contained 200 mM potassium phosphate buffer pH 8.0, 5 mM p-hydroxyphenylpyruvate (HPP), 10% ethanol (50 mM p-hydroxyphenylpyruvate stock solution in 96% ethanol was used), 44 mM ascorbate, 40 mM L-glutamate, 40 mM NAD⁺, 0.1 mM pyridoxal phosphate, and cell free extract of example 30 leading to a final concentration of 0.6 mg/ml soluble protein.

The assay was started by the addition of HPP and stopped after 65 h at 30° C. by the addition of 0.1 ml 1 N HCl to an aliquot of 0.5 ml of the reaction system. The samples were analyzed by HPLC as described in example 3.

The amounts of phydroxymandelate (HMA), p-hydroxyphenylglyoxylate (HPGL), and D-HPG produced in mg/l are summarized in Table 1.

TABLE 1

| Plasmid | HMA | HPGL | D-HPG |
|---|---|---|---|
| pJF-Sc-HmaS - Sc-HmaO - HpgAT | 17 | 67 | 9 |
| pJF-Ao-HmaS - Sc-HmaO - HpgAT | 0 | 166 | 11 |
| pJF-Sc-HmaS - mdIB - HpgAT | 37 | 73 | 24 |
| pJF-Ao-HmaS - mdIB - HpgAT | 0 | 97 | 26 |
| pJF119EH | 0 | 0 | 0 |

As can be seen, none of these compounds (HMA, HPGL and D-HPG) was detected in control experiments with cell free extracts derived from E. coli DH5α/pJF119EH.

### Example 32

### In Vitro Production of D-pG from phenylpyruvate

The assay mixture of 3 ml contained 200 mM potassium phosphate buffer pH 8.0, 5 mM phenylpyruvate (PP), 44 mM ascorbate, 40 mM L-glutamate, 40 mM NAD⁺, 0.1 mM pyridoxal phosphate, and cell free extract of example 30 leading to a final concentration of 0.3 mg/ml soluble protein.

The assay was started by the addition of PP and stopped after 39 h at 30° C. by the addition of 0.1 ml 1 N HCl to an aliquot of 0.5 ml of the reaction system. The samples were analyzed by HPLC as described in example 3.

Within 39 h 32 mg/l D-pG were produced with cell free extracts derived from E. coli DH5α/pJF-Ao-HmaS-Sc-HmaO-HpgAT, while 27 mg/l D-pG were produced with cell free extracts derived from E. coli DH5α/pJF-Ao-HmaS-mdlB-HpgAT. No D-pG was detected in control experiments with cell free extracts derived from E. coli DH5α/pJF 119EH.

### Example 33

### Construction of Plasmid pCR-BI-tyrA

The tyrA ORF encoding the E. coli chorismate mutase-prephenate dehydrogenase including the original RBS of E. coli (nucleotides 4740-5877 of accession AE000346) was amplified using
5′-GCGTGGAAGCTTAAGAGGTTTATTATGGTTGCTGAA-3′ [SEQ ID: No.35] (with Hind III restriction site underlined, and start codon double underlined) and
5′-GTGCACGGTCTCGAGCTGAATTCTTACTGGCGATTGTCAT-3′ [SEQ ID: No.36] (with Bsa I recognition and cleavage site underlined, and stop codon double underlined) as primers, and chromosomal DNA of the wild type E. coli strain LJ10 (Zeppenfeld et al 2000) as a template. Correct size (1169 bp) of the amplified fragment was confirmed by agarose gel electrophoresis.

The amplified fragment was directly inserted, according to the instruction of the supplier, into the vector pCR®-Blunt 11-TOPO® (Invitrogen, Groningen, The Netherlands) and transformed in chemically competent E. coli Top10 (Invitrogen, Groningen, The Netherlands). The transformants were selected on LB agar plates containing 50 mg/l kanamycin. A plasmid showing the correct insert sequence (as confirmed by sequencing) was called pCR-BI-tyrA and used for further investigations.

### Example 34

### Construction of plasmid pJF—Sc-HmaS-mdlB-HpgAT-tyrA

As a host strain for D-HPG production E. coli KB532 (Δ(pheA-tyrA), ΔtyrR, aroF^{fbr}, thiA, hsdR17, endA1, supE44) was chosen. KB532 is a L-tyrosine and L-phenylalanine auxotrophic strain. It lacks the genes for tyrA (chorismate mutase/prephenate dehydrogenase), pheA (chorismate mutase/prephenate dehydratase), and for the global regulator tyrR, and carries aroF^{fbr} encoding a feed back (L-tyrosine) resistant DAHP synthase. Overexpressing the tyrA gene in KB532 leads to a tyrosine producing E. coli strain, and therewith also to a strain delivering higher amounts of p-hydroxyphenylpyruvate.

To enable the overexpression of tyrA together with the artificial D-(H)PG operon, the cloned tyrA gene of example 33 was subcloned in plasmid pJF-Sc-HmaS-mdlB-HpgAT of example 28. The tyrA gene together with its RBS in pCR-BI-tyrA was excised from the expression vector by digestion with Hind III and Bsa 1, and the DNA fragment containing the tyrA ORF was purified by gel electrophoresis.

Partial digestion of plasmid pJF-Sc-HmaS-mdlB-HpgAT with Hind III was performed to obtain fragments of the linearised plasmid, because pJF-Sc-HmaS-mdlB-HpgAT contains three Hind III sites. Correct size of the restriction fragments was confirmed by agarose gel electrophoresis and the fragments of 8912 bp corresponding with the linearised plasmid were purified from the gel. After treatment with alkaline phosphatase to prevent recircularisation of the plasmid, these 8912 bp fragments were ligated with the tyrA Hind III/Bsa I fragment.

Recombinant plasmids were selected by complementation by transformation in the tyrA-deficient E. coli strain, KB532 (see above) and growth on minimal medium supplemented with 50 mg/l L-phenylalanine and 0.01 mM IPTG. Different transformants were selected, plasmid minipreps prepared, and the plasmid DNA analyzed by restriction mapping. A plasmid comprising the four genes in the correct order and direction in pJF 119EH was designated pJF-Sc-HmaS-mdlB-HpgAT-tyrA, the appropriate strain was called KB532/pJF-Sc-HmaS-mdlB-HpgAT-tyrA and used for further investigation.

### Example 35

### Fermentative Production of D-HPG

The D-HPG production of E. coli KB532/pJF-Sc-HmaS-mdlB-HpgAT-tyrA from glucose was investigated in mineral medium. This mineral medium consisted of Na citrate.3H₂O (1.0 g·l⁻¹), MgSO₄.7H₂O (0.3 g·l⁻¹), KH₂PO₄ (3.0 g·l⁻¹), K₂HPO₄ (12.0 g·l⁻¹), NaCl (0.1 g·l⁻¹), (NH₄)₂SO₄ (5.0 g·l⁻¹), CaCl₂.2H₂O (15.0 mg·l⁻¹), FeSO₄.7H₂O (75.0 mg·l⁻¹), thiamine.HCl (vitamin BI) (5.0 mg·l⁻¹), and L-phenylalanine (0.05 g·l⁻¹). Additional minerals were added in the form of a trace element solution (1 ml-1-1), which trace element solution was composed of Al₂(SO₄)₃.18H₂O (20.0 g·l⁻¹), CoCl₂.6H₂O (0.7 g·l⁻¹), CuSO₄.5H₂O (2.5 g·l⁻¹), H₃BO₃ (0.5 g·⁻¹), MnCl₂.4H₂O (20.0 g·l⁻¹) Na₂MoO₄.2H₂O (3.0 g-li), NiSO₄.6H₂O (2.0 g·l⁻¹), ZnSO₄.7H₂O (15.0 g·l⁻¹). A stock solution of Glucose (30 g-1-1) was autoclaved separately and added to the sterilized medium to a final concentration of 4 g·l⁻¹.

A single colony of the E. coli KB532/pJF-Sc-HmaS-mdlB-HpgAT-tyrA was used to inoculate 10 ml of minimal medium containing ampicillin (100 μg/ml) and incubated at 30° C. for 16 hours. 4 ml of this culture was subsequently used to inoculate 50 ml of the same medium and incubated at 33° C. and 180 rpm for 24 h. After 12 h at an OD₆₂₀ nm of 0.6, the cells were induced by adding 0.1 mM IPTG. A sample of the culture supernatant was adjusted to pH 5.8, lyophilized, and redissolved in D₂O. 600 MHz ¹H-NMR at 323 K showed the expected resonance spectrum and spiking with a small amount of HPG confirmed the presence of HPG. Redundant evidence was obtained from a 2-D COSY experiment. The amount present was determined to be 5 mg/l. (Besides HPG, 80 mg/l of its precursor p-hydroxyphenylglyoxylate was present, but no p-hydroxymandelate.)

1. A fermentative process in which a recombinant microorganism produces a D-phenylglycine product, in particular D-phenylglycine (D-pG) or D-p-hydroxyphenylglycine (D-HPG), wherein
a) for the production of D-pG, respectively for the production of D-HPG, phenylpyruvate (PP), respectively p-hydroxyphenylpyruvate (HPP) is withdrawn from the aromatic amino acid pathway
b) and is converted to mandelic acid (MA) or p-hydroxymandelic acid (HMA), respectively,
c) thereafter being converted into phenylglyoxylate or p-hydroxyphenylglyoxylate, respectively,
d) and the phenylglyoxylate or p-hydroxyphenylglyoxylate thereafter being converted into D-phenylglycine (D-pG) or D-p-hydroxyphenylglycine (D-HPG) by the action of a stereo-inverting D-aminotransferase, respectively.

2. A process according to claim 1, wherein the microorganism applied provides phenylpyruvate (PP) or p-hydroxyphenylpyruvate (HPP) at increased availability.

3. A process according to claim 2, wherein phenylpyruvate (PP) or p-hydroxyphenylpyruvate (HPP) is provided at increased availability by feeding phenylalanine (for PP) or tyrosine (for HPP), respectively.

4. A process according to any of claims 1-3, wherein the conversion of phenylpyruvate (PP) to mandelic acid (MA) or the conversion of p-hydroxyphenylpyruvate (HPP) to p-hydroxymandelic acid (HMA), occurs via a one-step enzymatic reaction catalyzed by a p-hydroxymandelate synthase.

5. A process according to any of claims 1-3, wherein the conversion of phenylpyruvate (PP) to mandelic acid (MA) or the conversion of p-hydroxyphenylpyruvate (HPP) to p-hydroxymandelic acid (HMA) occurs via a multi-step reaction, comprising the steps of converting PP or HPP to phenylacetaldehyde or p-hydroxyphenylacetaldehyde, then to phenylacetate or p-hydroxyphenylacetate, and finally to MA or HMA, respectively.

6. A process according to any of claims 1-5, wherein the conversion of hydroxymandelic acid (HMA), respectively of mandelic acid (MA), to p-hydroxyphenylglyoxylate, respectively to phenylglyoxylate is catalyzed in the presence of an enzyme, selected from a group of enzymes, consisting of mandelate dehydrogenase, p-hydroxymandelate dehydrogenase, oxygen dependent mandelate oxidases, and oxygen dependent p-hydroxymandelate oxidases.

7. A recombinant cell which is capable of secreting detectible amounts of D-HPG or D-pG and which contains
a) genes coding for enzymes which catalyze the conversion of p-hydroxyphenylpyruvate (HPP) to p-hydroxymandelic acid (HMA) or phenylpyruvate (PP) to mandelic acid (MA)
b) genes coding for enzymes which catalyze the conversion of p-hydroxymandelic acid (HMA) to p-hydroxyphenylglyoxylate or of mandelic acid (MA) to phenylglyoxylate
c) and genes coding for a stereo-inverting D-aminotransferase which catalyzes the conversion of p-hydroxyphenylglyoxylate to D-HPG or phenylglyoxylate to D-pG.

8. A recombinant plasmid containing a gene having a sequence corresponding with [SEQ ID: No.21] for the stereo-inverting conversion of phenylglyoxylate into D-pG, or of p-hydroxyphenylglyoxylate into D-HPG, and optionally at least one gene having a sequence corresponding with any of [SEQ ID: No.9] for the conversion of PP into MA, or of HPP into HMA, and/or [SEQ ID: No.15] for the conversion of MA into phenylglyoxylate, or of HMA into p-hydroxyphenylglyoxylate, as disclosed in this application, or the respective homologues of any of these sequences having at least 80%, preferably at least 90%, most preferably at least 95% homology with any of such sequences, for use in a process as claimed in claim 1.

9. A process according to any of claims 1-6, wherein the process is carried out in Escherichia coli as a host microorganism.

10. A recombinant cell according to claim 7, wherein the cell is an Escherichia coli cell.
